(19) United States
(12) Patent Application Publication
(10) Pub. No.: US 2010/0099758 A1
(43) Pub. Date: Apr. 22, 2010

(54) Regenerative wound healing using copper-silver citrate composition

(75) Inventors: William John Martin, South Pasadena, CA (US); Stephen Benet Palmer, Saint Louis, MO (US)

Correspondence Address: William John Martin, 1634 Spruce Street, South Pasadena, CA, 91030, US

(73) Assignee: MI Hope Inc., South Pasadena, CA (US)

(21) Appl. No.: 12/288,749

(22) Filed: Oct. 22, 2008

(51) Int. Cl. A61K 31/30; A61P 17/02

(52) U.S. Cl. 514/495

(57) ABSTRACT

A composition containing copper and silver ions electrolytically generated in a solution of citric acid that also contains potassium carbonate is shown to expedite the healing of wounds with regeneration of normal tissue architecture and without excessive scarring. A wide range of wounds and other forms of tissue damage affecting the skin and other areas of the body respond favorably to the direct application of the composition, with expedited healing, re-growth of normal appearing (non-scar) tissue and symptomatic relief from pain and swelling. The composition acts in both humans and animals through a novel mechanism provisionally termed the tissue regeneration induced pathway (TRIP).

### CROSS REFERENCE TO RELATED PATENT APPLICATIONS

This patent is an extension of a pending patent application “Antibacterial composition and method of production.” Martin W J and Palmer S. Submitted Dec. 9, 2007

### RELATED UNITED STATES ISSUED PATENTS

U.S. Pat. No. 4,287,184 Process for healing wounds

U.S. Pat. No. 4,604,234 Protein having cell growth stimulating action, composition thereof and method for producing the same

U.S. Pat. No. 4,861,757 Wound healing and bone regeneration using PDGF and IGF-I

U.S. Pat. No. 5,461,030 Compositions and methods for enhancing wound healing

U.S. Pat. No. 6,689,351 Use of GM-CSF to promote accelerated wound healing

U.S. Pat. No. 6,998,109 Emu-based formulations with lidocaine for wound treatment by inhibiting microbial activity

U.S. Pat. No. 7,118,761 Method for producing a silver-containing wound care device

U.S. Pat. No. 7,166,549 Antimicrobial, anti-inflammatory, wound-healing and disinfecting glass and use thereof

U.S. Pat. No. 7,271,187 Compositions and methods for promoting tissue regeneration

U.S. Pat. No. 7,312,198 Protein compositions for promoting wound healing and skin regeneration

U.S. Pat. No. 7,407,953 Wound healing

U.S. Pat. No. 4,760,051 Use of GHL-Cu as a wound-healing and anti-inflammatory agent

U.S. Pat. No. 4,292,324 Aqueous zinc solutions for medical use

U.S. Pat. No. 5,164,367 Method of using copper(II) containing compounds to accelerate wound healing

U.S. Pat. No. 6,761,729 Wound treatment method and device with combination of ultrasound and laser energy

U.S. Pat. No. 7,016,737 Method and device for wound healing

### RELATED UNITED STATES PENDING PATENT APPLICATIONS

20070009611 Treatment of wounds and compositions employed

20070128296 Reduction of reactive oxygen species in chronic wound management

20080249041 Formulations comprising antisense nucleotides to connexins

20080247995 Wound and cutaneous injury healing with a nucleic acid encoding a proteoglycan polypeptide

20080241210 Wound healing agent and composition

20080242631 Impaired wound healing compositions and treatments

### FIELD OF THE INVENTION

Wound healing

### REFERENCES TO PUBLISHED LITERATURE RELATING TO WOUND HEALING

General Articles

Enoch S, and Leaper DJ. Basic science of wound healing. Surgery (Oxford) 6: 31-37, 2008

Williamson D, and Harding K. Wound healing. Medicine 32: 4-7, 2004.

Rhett J M, Ghatnekar G S, Palatinus J A, O'Quinn M, Yost M J and Gourdie R G. Novel therapies for scar reduction and regenerative healing of skin wounds. Trends in Biotechnology 26: 173-180, 2008

Copper Related:

Gadi Borkow G, Gabbay J, and Zatcoff RC. Could chronic wounds not heal due to too low local copper levels. Medical Hypotheses 70: 610-613, 2008.

Sen C K, Khanna S, Venojarvi M, et al., Copper-induced vascular endothelial growth factor expression and wound healing, Am J Physiol Heart Circ Physiol 282: H1821-H1827, 2002.

Mandinov L, Mandinova A, Kyurkchiev S, et al., Copper chelation represses the vascular response to injury, Proc Natl Acad Sci USA 100: 6700-6705, 2003

Soldi M R, Graziani I, et al. S100A13 mediates the copper-dependent stress-induced release of IL-1alpha from both human U937 and murine NIH 3T3 cells, J Cell Sci 116: 2687-2696, 2003.

Cunningham J J, Leffell M, and Harmatz P. Burn severity, copper dose, and plasma ceruloplasmin in burned children during total parenteral nutrition, Nutrition 9: 329-332, 1993.

Borkow G, and Gabbay J. Copper as a biocidal tool, Curr Med Chem 12: 2163-2175, 2005.

Pickart et al., “Growth-Modulating Tripeptide (glycylhistidyllysine): Association with Copper and Iron in Plasma and Stimulation of Adhesive and Growth of Hepatoma Cells in Culture by Tripeptide-Metal Ion Complexes,” J. Physiol. 102:129-139, 1980.

Pickart et al., Growth-Modulating Plasma Tripeptide May Function By Facilitating Copper Uptake Into Cells. Nature 288:715-717, 1980.

Williams et al.,Glycyl-L-Histidyl-L-Lysine, a Growth Promoting Factor for Human Cells, Cytobios 27: 19-25, 1980.

Khorasani G, Hosseinimehr S J, and Kaghazi Z. The alteration of plasma's zinc and copper levels in patients with burn injuries and the relationship to the time after burn injuries. Singapore Med J. 49:627-30, 2008.

Cunningham J J, Martha K. Lydon R N, Emerson R, and Harmatz P R. Low ceruloplasmin levels during recovery from major burn injury: Influence of open wound size and copper supplementation Nutrition 12: 83-88, 1996.

Wound Healing and Energy Medicine

Funk R H W, Monsees T, and Özkucur N. Electromagnetic effects—From cell biology to medicine. Progress in Histochemistry and Cytochemistry (in Press).

Becker R O. The body electric: electromagnetism and the foundation of life, 1985. ISBN:0-688-00123-8.

Becker R O. The bioelectric factors in amphibian-limb regeneration, J Bone Jt Surg Am 43-A: 643-656, 1961.

Martin W J. Etheric biology. Exp Mol Pathol. 78: 221-227, 2005

### STATEMENT REGARDING FEDERALLY SPONSORED RESEARCH OR DEVELOPMENT

No Federal funding was received in support of research covered in this patent application.

### BACKGROUND OF THE INVENTION

The present invention stems from a series of serendipitous observations of expedited healing of skin wounds without significant scarring, in both humans and animals following the application of a copper-silver citrate composition. The composition was initially developed as an effective environmental antimicrobial disinfectant reagent. It has now been shown to promote wound healing and tissue regeneration.

The healing of wounds proceeds along various biological pathways that are influenced by numerous factors, many of which are still poorly understood. Inflammation is evoked by traumatic injuries and can be greatly enhanced if the resulting wound becomes the site of microbial invasion. As the inflammation subsides, proliferating fibroblasts enter the wound site to assist in the closure of any remaining discontinuity of the tissue. This closure is largely achieved by the production of collagen by the fibroblasts, essentially leading to scarring of the tissue. The scarring is generally considered a barrier to the full regeneration of more specialized types of cells and to a complete regaining of normal function. The origins of the regenerating specialized cells can include both existing cells in the vicinity of the wound and newly arriving stem cells that enter the wound from the circulation.

Impaired healing can consist of i) persisting inflammation, ii) non-union of the tissue edges and iii) excessive scarring without restoration of normal structure and function. Many efforts have been made to overcome such impairments. These include the suppression of any residual microbial contamination; excision of necrotic tissue; promotion of fibroblast growth in the case of non-union; inhibition of fibroblast growth in an attempt to minimize scarring; and means of potentially promoting the growth of specialized cells to achieve a complete regeneration of the wounded tissue. Most of the present day studies in wound healing are directed at manipulating the chemical signals that regulate the biochemical activities of cells. There is much emphasis on secreted growth factors and on a class of molecules called connexins that pass directly between cells. Many of these approaches are covered in the cited awarded and pending patents.

Restoring any local or systemic nutritional deficiencies of vitamins and minerals has provided some positive results in limited studies, mainly performed in developing countries. The systemic administration of copper ions linked to a tripeptide is covered in patents U.S. Pat. Nos. 4,760,051 and 5,164,367.

The capacity of amphibian and reptiles to regenerate severed limbs provides a hopeful indication that similar success can potentially occur in higher animals including humans. It is interesting that the tissue regeneration in more primitive animals can seemingly involve neuro-electrical pathways, possibly through the induction of dedifferentiation of mature cells. Even in humans, electrical and ultrasound derived energies appear to have wound healing promoting activities (e.g. U.S. Pat. Nos. 7,016,737 and 6,761,729). One of the inventors has considerable evidence that nerves may be a source of an alternative cellular energy that is distinct from that obtained by oxidative phosphorylation of food. The inventors were therefore open to the suggestion that non-conventional means may be available to affect tissue repair in humans. They, therefore, pursued the initial reports of seemingly very striking tissue repair occurring from the application of a copper-silver citrate solution.

Copper citrate was developed as an antibacterial product that could show some preferential killing of gram positive bacteria It is produced by electrolysis of copper to a concentration of 300-500 ppm in a citrate solution, as a chelator. It was further decided to include silver at a concentration of approximately 50 ppm to potentially allow for better conductivity and possibly a broader antimicrobial action. The solution has performed very well as a disinfectant in hospitals and other locations. It has also displayed somewhat remarkable wound healing properties.

### BRIEF DESCRIPTION OF THE DRAWINGS(FIGURES)

None

### BRIEF SUMMARY OF THE INVENTION

The invention provides a copper-silver-citrate solution that has wound healing activities. Although the mechanism of its action is not well understood, the observations made t6 date far exceed anything reported in the current literature. The healing of skin wounds has been documented in both animals and humans and requires no more that daily exposure to a wounded area to achieve healing. More importantly, skin and other lesions are healing with far less scarring than would be expected from allowing the natural healing process to proceed within the intervention. Various concentrations of the stock solution have been tried with possibly more effect when undiluted but clearly with an effect even at a 10% concentration. Several examples are cited below.

### DETAILED DESCRIPTION OF THE INVENTION

The product is routinely produced by electrolysis in a 3-5% citric acid solution buffered with potassium carbonate to pH 5.5. The current is provided by a 200 Amp 12 volt battery. The amount of silver dissolved is calculated to be between 30 and 50 ppm. At the end of the silver electrolysis, the pH is again adjusted to 5.5 and then copper is electrolytically dissolved to a concentration between 300-500 ppm. The pH of the solution is then adjusted to between 2.5 and 3.5 using additional citric acid. It is then filtered and bottled. Several batches have been tested and all have shown wound healing activities. Ongoing nuclear magnetic resonance (NMR) studies are consistent with a close association of an enlarged copper atom with the central carboxylic functional group of the citrate molecule.

Individual L.H. was using an electric screwdriver and inadvertently held his thumb in the path of the screw. The screw passed through both the front and the nail side of his thumb, leaving a gaping hole surrounded by flesh. It was apparent that the bone must have been penetrated. Two days elapsed with the thumb becoming more painful and beginning to throb. Because of concern about possible infection, the copper-silver-citrate solution was sprayed onto the wound at 100% concentration. Apart from the stinging, there were no adverse effects. The individual was instructed to dip his thumb into a 2 oz container of the copper-silver-citrate solution twice daily for 7 days. The hole closed over in one day and the swelling had resolved. By day 3 the wound had healed without noticeable scabbing, discolorization or scarring. An ultrasound of his thumb at day 7 showed a fully intact terminal phalange (finger bone). His thumb is now fully functional and unscarred with a normal appearing thumbprint.

Individual R.B. had a long standing deep wound involving his left heel. His foot had been operated upon approximately a year earlier because of a methicillin resistant Staphylococcus aureus (MRSA) infection. The surgery included removal of a portion of the calcaneus (heel) bone. He was still left with an open, draining wound that required daily dressings. In July 2008, he began to use the 100% solution as a twice daily spray and quickly noticed far less pain and drainage. His wound has become noticeably smaller and seemingly the excised part of the calcaneus bone has begun to fill in. The remaining wound is now quite shallow with little drainage, allowing him to walk with only minimal pain. He has switched to using the 20% solution and his physicians are anticipating full recovery.

Individual J.B. had severe sunburn over his arms, neck and back with blister formation.. He became pain free within an hour of his wife spraying a 20% solution onto the affected areas. Interestingly, there was no subsequent peeling of the severely sun burnt areas. The same individual developed a tooth abscess for which he sought medical and dental advice. His was able to see his physician but the dental appointment was scheduled for much later. His physician confirmed the abscess and rescheduled him to return in a few days. He took some of the copper-silver-citrate solution into his mouth in order to bathe the abscess for about 3 minutes. The pain ceased soon after the first treatment. He repeated the procedure twice daily and by the end of the second day, the swelling had gone. He cancelled his scheduled physician appointment and when he finally saw his dentist, he was told his tooth was normal and there was no need for an extraction. Similarly, individual D.W. experienced complete recovery from a dental abscess by exposing his abscessed tooth to the solution for several days. He thereby avoided the need for a previously scheduled root canal surgery. What is most remarkable about these observations is the apparent healing of the underlying tooth pathology that lead to the abscess formation. This is an extremely rare occurrence in routine dentistry.

The animal experience includes a dog belonging to individual C.S. that was treated for yeast and tropical Staphylococcus infection of its ears and abdomen. The skin in these areas was broken and inflamed. Three applications of a 20% copper-silver-citrate solution were made to the affected areas. Not only was there relief from the infection, as was expected from the product's antibacterial action, but the skin areas showed near complete restoration to normal appearance. C.S. commented that she was “extremely impressed by results.”

Another dog belonging to individual L.H. had infected and weeping areas on numerous parts of its body. These areas cleared up without residual scarring within 6 days of daily applying 100% solution.

Since the copper-silver-citrate solution was originally intended as a disinfectant for MRSA, it has been tried on several animals with skin infections. A goat breeder began to use the copper-silver-citrate solution as an environmental disinfectant in the hope of controlling an outbreak of MRSA infections among her herd of goats. A veterinarian was prepared to perform euthanasia on a severely infected goat and also on an infected dog. Both animals were essentially cured the night before the planned euthanasia by a single application of a 20% solution to the infected skin lesions. The veterinarian could find no signs of infection the next day in either animal. He requested some copper-silver-citrate solution for use as an internal irrigation disinfectant during a caesarian section to be performed on a pregnant goat. The operation was successful and seven offspring (kids) were delivered. The copper-silver-citrate solution was also applied twice daily to the site of surgical excision. Surprisingly, the mother goat was up and running by the next day. When examined by the veterinarian at 7 days post operatively, he concluded there were no internal adhesions, a common complication of caesarian section performed in goats. Even more remarkable, the surgical scar was quite unlike that expected a week after surgery. Instead, the veterinarian remarked that it looked years old. Two weeks later after three more applications of 20% solution the scar was gone and fur had grown back over the incision area. As a measure of the health of this animal, all 7 of her kids survived, which is rather rare for caesarian section delivered goats.

In addition to these successes, several other individuals have used the spray on a variety of unhealed skin lesions, minor burns, allergy (poison oak) rash and even on skin blemishes, including an area of cellulite. Most individuals used 20% solution, while some simply used a 10% solution. All of the individuals with painful skin lesions experienced pain relief soon after the application of the solution. The presumptive poison oak rash disappeared within an hour with no residual swelling or itchiness. The appearances of various skin blemishes, including the cellulite, have also been improved using daily application of the solution without any indications of toxicity.

A striking characteristic seen in these studies is the lack of scarring of the affected areas indicative of a regeneration rather than simply of wound closing effect. Moreover, except for the heel lesion that is still being treated, complete healing is being achieved. Although, not bound by the model, the results are consistent with a true regenerative process. The process is rapid and seemingly not due to inhibition of fibroblast growth, but rather to an expedited regeneration of specialized cells. Instead, the copper-silver-citrate composition appears to be directly facilitating a natural healing process.

Copper citrate is thought to be mainly lethal to gram positive bacteria mainly through its chelating activity of iron (previous patent application by the inventors). Copper can also be a co-factor for some cellular enzymes. There are initial indications; however, that copper citrate without the presence of silver ions is less effective than is the copper-silver-citrate combination. This observation provides some support to the suggestion that the copper and silver atoms and citrate molecule may be cooperatively functioning in some forms of energy transmission. The potassium carbonate in the copper-silver-citrate solution may also be participating in an overall reaction. A working model is that one or more of these components is oscillating between different configurations and this oscillation mirrors or possibly transmits a natural communication and energy generating system employed by the body in response to various needs including tissue regeneration.

Although, in no way bound by this speculative explanation, it is certainly conceivable that the copper-silver-citrate solution is facilitating the spread of some form of electrical or other energy signaling that promote the regeneration of specialized cells within recovering wounds, thereby reestablishing normal tissue architecture. It is to be expected that alternative approaches to engaging and transmitting this hypothetical “tissue regeneration induction pathway” (TRIP) will emerge from the studies being performed on the current composition. Indeed, the copper-silver-citrate composition will provide a prototype or template system to characterize the proposed tissue regeneration induction pathway and lead to the development of other products and procedures that act through a similar wound healing mechanism.

Whatever the actual mechanism, it is clear that the underlying principle and actual application of the invention will be of immediate benefit in the treatment of skin and deeper wounds, as well as many other conditions.

The examples of wound healing cited in this application are not meant in any way to be limiting to the potential use of copper-silver-citrate and related formulations to enhance the rejuvenating capacity of various cell types. As the mechanism of action of the current composition becomes characterized, many additional approaches to improving wound healing and tissue regeneration that are based on the same mechanism will become apparent to those in the field. Indeed, it is anticipated that knowledge of the findings with the present composition will open a new field of studies on tissue regeneration. Predictably, many types of alternative and derivative products will ensure from the present invention, as will other procedures that are not necessarily based on the use of a pharmaceutical preparation. It is the intent of this application to obtain patent protection for all such alternative and derivative products as well as non-pharmaceutical based procedures that arise from the invention disclosed in this application.

It is also to be emphasized that the various compositions can potentially be applied to internal organs, such as the heart, liver, brain and spinal cord. They may also have activities on amputated limbs. Additional advantages and modifications will readily occur to those skilled in the art of efforts towards promoting and stimulating the process of wound healing and tissue regeneration. Variations and changes may be made without departing from the spirit and principle of the invention. For example, rather than using a solution, it may be more suitable in some circumstances to use the composition in powder, liniment, gel, lotion, cream, ointment or suppository form.

What we claim as our invention is:

1. A pharmaceutical composition comprising a solution of copper-silver-citrate plus potassium carbonate at a sufficient concentration, which when applied to a wound is able to achieve one or more of the following benefits: faster healing of the wound; less scarring of the wound; more rapid and complete growth of specialized (non-fibroblast) cells within the wound; improved cosmetic appearance of the wound; and greater symptomatic relief; than would have occurred if the composition had not been applied to the wound.

2. A method of treating a wound comprising the application of the pharmaceutical composition of claim 1 to a wound in order to achieve one or more of the following benefits: faster healing of the wound; less scarring of the wound; more rapid- and complete growth of-specialized (non-fibroblast) cells within the wound;
improved cosmetic appearance of the wound; and greater symptomatic relief; than would have occurred if the composition had not been applied to the wound.

3. Pharmaceutical compositions and procedures that achieve one or more of the benefits listed in claim 1 through the mechanism of action that is directly comparable to the biological activity of the composition of claim 1 and whose development has or would have been been assisted by the findings obtained in studies on the composition of claim 1 or on related products that are derived as a result of such studies.

4. The method of claim 2, wherein the wound is a lesion caused by either acute or chronic infection of a part or region of the body including the skin and internal organs of a human or animal.

5. The method of claim 2, wherein the wound is a lesion caused by either acute or chronic trauma to a part or region of the body including the skin or internal organs of a human or animal.

6. The method of claim 2, wherein the wound is a lesion caused by either acute or chronic deficiency in the blood supply to a part or region of the body including ischemic ulceration resulting from arterial obstruction or from pressure as is the case of bedsores.

7. The method of claim 2, wherein the wound is selected from the group consisting of burns caused by the sun, other hot objects and substances or chemicals.

8. The method of claim 2, wherein the wound is the result of surgery, including the skin incision and incisions performed within the internal structures of the body of a human or animal.

9. The method of claim 2, wherein the wound is selected from the group consisting of allergic reactions, autoimmune diseases and responses to toxins that involve the skin or internal structures of the body of a human or animal.

10. The method of claim 2, wherein the target of therapy is a skin blemish including cellulite.

11. The method of claim 2, wherein the target of therapy is a skin lesion is of a proliferative nature, including psoriasis and cancers.

12. The method of claim 2, wherein the wound is a tooth abscess or other inflammatory lesion of the mouth.

13. The composition of claim 1 in which the pharmaceutical composition comprises a solution that contains copper at a concentration between 300 to 500 ppm and a concentration of silver between 30-50 ppm in a citrate solution

14. The composition of claim 1 in which the pharmaceutical composition comprises a solution that contains copper citrate at a concentration between 300 to 500 ppm in a citrate solution.

15. The composition of claim 1 in which the pharmaceutical composition comprises a solution that contains silver citrate at a concentration between 30 to 50 ppm in a citrate solution
